(19) United States
(12) Patent Application Publication
(10) Pub. No.: US 2010/0041871 A1
(43) Pub. Date: Feb. 18, 2010

(54) Preparation of Canola Protein Isolate from Canola Oil Seeds ("Blendertein")

(76) Inventors: Kevin I. Segall, Winnipeg (CA); Brent E. Green, Warren (CA); Martin Schweizer, Winnipeg (CA)

Correspondence Address: SIM & MCBURNEY, 330 UNIVERSITY AVENUE, 6TH FLOOR, TORONTO, ON, M5G 1R7, CA

(21) Appl. No.: 12/542,931

(22) Filed: Aug. 18, 2009

Related U.S. Application Data
(60) Provisional application No. 61/136,192, filed on Aug. 18, 2008.

(51) Int. Cl. C07K 1/14; C07K 14/415

(52) U.S. Cl. 530/377

(57) ABSTRACT

Canola protein isolate is recovered from canola oil seeds by crushing the oil seeds and extracting the crushed canola oil seeds. Fat co-extracted from the crushed oil seeds is removed from the aqueous canola protein solution which then is processed by the micellar route to obtain the canola protein isolate.

### REFERENCE TO RELATED APPLICATION

This application claims priority under 35 USC 119(e) from U.S. Provisional Patent Application No. 61/136,192 filed Aug. 18, 2008.

### FIELD OF INVENTION

The present invention relates to the preparation of a canola protein isolate.

### BACKGROUND TO THE INVENTION

In the processing of canola oil seeds, the seeds are crushed to remove most of the canola oil component of the seeds. The residual crushed seeds are solvent extracted, usually using hexane, to recover the remainder of the oil. The solvent then is recovered for reuse to produce a canola oil seed meal.

Canola oil seed protein isolates having protein contents of at least 100 wt % (N×6.25) can be formed from oil seed meal by a process as described in copending U.S. patent application Ser. No. 10/137,391 filed May 3, 2002 (U.S. Patent Application Publication No. 2003-0125526 A1 and WO 02/089597) and U.S. patent application Ser. No. 10/476,230 filed Jun. 9, 2004 (U.S. Patent Application Publication No. 2004-0254353 A1), assigned to the assignee hereof and the disclosures of which are incorporated herein by reference. The procedure involves a multiple step process comprising extracting canola oil seed meal using an aqueous salt solution, separating the resulting aqueous protein solution from residual oil seed meal, increasing the protein concentration of the aqueous solution to at least about 200 g/L while maintaining the ionic strength substantially constant by using a selective membrane technique, diluting the resulting concentrated protein solution into chilled water to cause the formation of protein micelles, settling the protein micelles to form an amorphous, sticky, gelatinous, gluten-like protein micellar mass (PMM), and recovering the protein micellar mass from supernatant having a protein content of at least about 100 wt % (N×6.25). As used herein, protein content is determined on a dry weight basis. The recovered PMM may be dried.

In one embodiment of the process, the supernatant from the PMM settling step is processed to recover canola protein isolate from the supernatant. This procedure may be effected by initially concentrating the supernatant using an ultrafiltration membrane and drying the concentrate. The resulting canola protein isolate has a protein content of at least about 90 wt %, preferably at least about 100 wt % (N×6.25).

The procedures described in U.S. patent application Ser. No. 10/137,391 are essentially batch procedures. In copending U.S. patent application Ser. No. 10/298,678 filed Nov. 19, 2002 (WO 03/043439), assigned to the assignee hereof and the disclosures of which are incorporated herein by reference, there is described a continuous process for making canola protein isolates. In accordance therewith, canola oil seed meal is continuously mixed with an aqueous salt solution, the mixture is conveyed through a pipe while extracting protein from the canola oil seed meal to form an aqueous protein solution, the aqueous protein solution is continuously conveyed through a selective membrane operation to increase the protein content of the aqueous protein solution to at least about 50 g/L, while maintaining the ionic strength substantially constant, the resulting concentrated protein solution is continuously mixed with chilled water to cause the formation of protein micelles, and the protein micelles are continuously permitted to settle while the supernatant is continuously overflowed until the desired amount of PMM has accumulated in the settling vessel. The PMM is recovered from the settling vessel and may be dried. The PMM has a protein content of at least about 90 wt % (N×6.25), preferably at least about 100 wt %. The overflowed supernatant may be processed to recover canola protein isolate therefrom, as described above.

The applicants are aware of procedures used to recover various proteins from oil seeds in which the oil seeds are ground and then processed to recover the protein. Representative examples are U.S. Pat. Nos. 2,762,820 and 4,151,310. Canola is not among the oil seeds processed in such prior art procedures.

Canola is also known as rapeseed or oil seed rape.

### SUMMARY OF INVENTION

In the process of the present invention, the initial oil removal step generally carried out on canola oil seeds is omitted. In accordance with one aspect of the present invention, there is provided a process for the preparation of a canola protein isolate from canola oil seeds, which comprises:

grinding canola oil seeds,

extracting the ground canola oil seeds with an aqueous extracting medium to solubilize canola protein in the ground canola oil seeds to form an aqueous canola protein solution,

separating the aqueous canola protein solution from residual ground canola oil seeds,

defatting the aqueous canola protein solution,

clarifying the defatted aqueous canola protein solution,

concentrating the clarified aqueous canola protein solution while maintaining the ionic strength substantially constant to form a concentrated canola protein solution,

optionally diafiltering the concentrated canola protein solution,

optionally pasteurizing the optionally diafiltered and concentrated canola protein solution,

diluting the concentrated canola protein solution into chilled water to cause the formation of canola protein micelles,

collecting the canola protein micelles as a protein micellar mass,

drying the protein micellar mass to form a canola protein isolate having a protein content of at least about 90 wt % (N×6.25) d.b., preferably at least about 100 wt % d.b., and

optionally processing supernatant from the collection of the protein micellar mass to form a further canola protein isolate having a protein content of at least about 90 wt % (N×6.25) d.b., preferably at least about 100 wt % d.b.

The procedure used herein to recover a canola protein isolate from canola oil seeds is superior to the process of recovering canola protein isolate according to the above-described processes, wherein the starting material is the residual meal from processing the canola oil seeds for the primary purpose of recovering the oil from the seeds, in that a higher quality product is obtained herein in terms of the colour of the isolate, i.e. lesser pigmentation.

The canola protein isolate produced according to the process herein may be used in conventional applications of protein isolates, such as, protein fortification of processed foods and beverages, emulsification of oils, body formers in baked goods and foaming agents in products which entrap gases. In addition, the canola protein isolate may be formed into protein fibers, useful in meat analogs, may be used as an egg white substitute or extender in food products where egg white is used as a binder. The canola protein isolate may be used as nutritional supplements. Other uses of the canola protein isolate are in pet foods, animal feed and in industrial and cosmetic applications and in personal care products.

### GENERAL DESCRIPTION OF INVENTION

In the present invention, intact canola oil seeds are ground to provide a ground mass of canola oil seeds. The initial step of the process of providing a canola protein isolate from the ground mass of canola oil seeds involves solubilizing proteinaceous material from the ground canola oil seeds. Alternatively, the seeds may be ground wet, using any convenient equipment, such as a high shear pump, to simultaneously grind the seed and solubilize the protein. The proteinaceous material recovered from canola seed may be the protein naturally occurring in canola seed or the proteinaceous material may be a protein modified by genetic manipulation but possessing characteristic hydrophobic and polar properties of the natural protein.

Protein solubilization is effected most efficiently by using a food grade salt solution since the presence of the salt enhances the removal of soluble protein from the crushed canola oil seeds. Where the canola protein isolate is intended for non-food uses, non-food-grade chemicals may be used. The salt usually is sodium chloride, although other salts, such as, potassium chloride, may be used. The salt solution has an ionic strength of at least about 0.05, preferably at least about 0.10, to enable solubilization of significant quantities of protein to be effected. As the ionic strength of the salt solution increases, the degree of solubilization of protein in the canola oil seed initially increases until a maximum value is achieved. Any subsequent increase in ionic strength does not increase the total protein solubilized. The ionic strength of the food grade salt solution which causes maximum protein solubilization varies depending on the salt concerned.

In view of the greater degree of dilution required for protein precipitation with increasing ionic strengths, it is usually preferred to utilize an ionic strength value less than about 0.8, and more preferably a value of about 0.1 to about 0.15.

In a batch process, the salt solubilization of the protein is effected at a temperature of from about 5° C. to about 75° C., preferably accompanied by agitation to decrease the solubilization time, which is usually about 10 to about 60 minutes. It is preferred to effect the solubilization to extract substantially as much protein from the canola oil seed meal as is practicable, so as to provide an overall high product yield.

The lower temperature limit of about 5° C. is chosen since solubilization is impractically slow below this temperature while the upper preferred temperature limit of about 75° C. is chosen due to the denaturation temperature of some of the present proteins.

In a continuous process, the extraction of the protein from the canola oil seed is carried out in any manner consistent with effecting a continuous extraction of protein from the canola oil seed. In one embodiment, the crushed canola oil seed is continuously mixed with a food grade salt solution and the mixture is conveyed through a pipe or conduit having a length and at a flow rate for a residence time sufficient to effect the desired extraction in accordance with the parameters described herein. In such continuous procedure, the salt solubilization step is effected rapidly, in a time of up to about 10 minutes, preferably to effect solubilization to extract substantially as much protein from the canola oil seed as is practicable. The solubilization in the continuous procedure is effected at temperatures between about 10° C. and about 75° C., preferably between about 15° C. and about 35° C.

The aqueous food grade salt solution generally has a pH of about 5 to about 6.8, preferably about 5.3 to about 6.2, the pH of the salt solution may be adjusted to any desired value within the range of about 5 to about 6.8 for use in the extraction step by the use of any convenient acid, usually hydrochloric acid, or alkali, usually sodium hydroxide, as required.

The concentration of ground canola oil seeds in the food grade salt solution during the solubilization step may vary widely. Typical concentration values are about 5 to about 25% w/v.

The protein extraction step with the aqueous salt solution has the additional effect of solubilizing the fats which are present in the canola seeds, which then results in the fats being present in the aqueous phase.

The protein solution resulting from the extraction step generally has a protein concentration of about 3 to about 40 g/L, preferably about 10 to about 30 g/L.

The aqueous salt solution may contain an antioxidant. The antioxidant may be any convenient antioxidant, such as sodium sulfite or ascorbic acid. The quantity of antioxidant employed may vary from about 0.01 to about 1 wt % of the solution, preferably about 0.05 wt %. The antioxidant serves to inhibit oxidation of phenolics in the protein solution.

The aqueous phase resulting from the extraction step then may be separated from the residual canola seed material, in any convenient manner, such as by employing a decanter centrifuge, followed by disc centrifugation to remove residual seed material. The separated residual seed material may be dried for disposal or further processing.

The fat present in the aqueous canola protein solution may be removed by a procedure as described in U.S. Pat. Nos. 5,844,086 and 6,005,076, assigned to the assignee hereof and the disclosures of which are incorporated herein by reference.

As described therein, the aqueous canola protein solution may be chilled to a temperature of about 3° to about 7° C., to cause fat to separate from the aqueous phase for removal by any convenient procedure, such as by decanting. Alternatively, the fat may be removed at higher temperatures by centrifugation using a cream separator. Once the fat has been removed, the aqueous canola protein solution may be further clarified by filtration. The canola oil recovered from the aqueous canola protein solution may be processed to use in commercial applications of canola oil.

Alternatively, the aqueous canola protein solution may be simultaneously separated from the oil phase and the residual canola seed material by any convenient procedure, such as using a three phase decanter. The aqueous canola protein solution may then be further clarified by filtration.

The colour of the final canola protein isolate can be improved in terms of light colour and less intense yellow by the mixing of powdered activated carbon or other pigment adsorbing agent with the separated aqueous protein solution and subsequently removing the adsorbent, conveniently by filtration, to provide a protein solution. Diafiltration also may be used for pigment removal.

Such pigment removal step may be carried out under any convenient conditions, generally at the ambient temperature of the separated aqueous protein solution, employing any suitable pigment adsorbing agent. For powdered activated carbon, an amount of about 0.025% to about 5% w/v, preferably about 0.05% to about 2% w/v, is employed.

As an alternative to extracting the ground canola oil seed with an aqueous salt solution, such extraction may be made using water alone, although the utilization of water alone tends to extract less protein from the ground canola oil seed than the aqueous salt solution. Where such alternative is employed, then the salt, in the concentrations discussed above, may be added to the protein solution after separation from the residual ground oil seed in order to maintain the protein in solution during the concentration step described below. When a first fat removal step is carried out, the salt generally is added after completion of such operations.

Another alternative procedure is to extract the ground canola oil seed with the food grade salt solution at a relatively high pH value above about 6.8, generally up to about 9.9. The pH of the food grade salt solution may be adjusted in pH to the desired alkaline value by the use of any convenient food-grade alkali, such as aqueous sodium hydroxide solution. Alternatively, the ground oil seed may be extracted with the salt solution at a relatively low pH below about pH 5, generally down to about pH 3. Where such alternative is employed, the aqueous phase resulting from the ground oil seed extraction step then is separated from the residual canola seed material, in any convenient manner, as discussed previously. The separated residual canola oil seed material may be dried for disposal or further processing.

The aqueous protein solution resulting from the high or low pH extraction step then is pH adjusted to the range of about 5 to about 6.8, preferably about 5.3 to about 6.2, as discussed above, prior to further processing as discussed below. Such pH adjustment may be effected using any convenient acid, such as hydrochloric acid, or alkali, such as sodium hydroxide, as appropriate.

The aqueous canola protein solution is concentrated to increase the protein concentration thereof while maintaining the ionic strength thereof substantially constant. Such concentration generally is effected to provide a concentrated protein solution having a protein concentration of about 50 to about 250 g/L, preferably to about 200 g/L.

The concentration step may be effected in any convenient manner consistent with batch or continuous operation, such as by employing any convenient selective membrane technique, such as ultrafiltration or diafiltration, using membranes, such as hollow-fibre membranes or spiral-wound membranes, with a suitable molecular weight cut-off, such as about 3,000 to about 100,000 daltons, preferably about 5,000 to about 10,000 daltons, having regard to differing membrane materials and configurations, and, for continuous operation, dimensioned to permit the desired degree of concentration as the aqueous protein solution passes through the membranes.

As is well known, ultrafiltration and similar selective membrane techniques permit low molecular weight species to pass therethrough while preventing higher molecular weight species from so doing. The low molecular weight species include not only the ionic species of the food grade salt but also low molecular weight materials extracted from the source material, such as, carbohydrates, pigments and anti-nutritional factors, as well as any low molecular weight forms of the protein. The molecular weight cut-off of the membrane is usually chosen to ensure retention of a significant proportion of the protein in the solution, while permitting contaminants to pass through having regard to the different membrane materials and configurations.

The concentrated protein solution then may be subjected to a diafiltration step using an aqueous salt solution of the same molarity and pH as the extraction solution. Such diafiltration may be effected using from about 2 to about 20 volumes of diafiltration solution, preferably about 5 to about 10 volumes of diafiltration solution. In the diafiltration operation, further quantities of contaminants are removed from the aqueous canola protein solution by passage through the membrane with the permeate. The diafiltration operation may be effected until no significant further quantities of contaminants and visible colour are present in the permeate. Such diafiltration may be effected using the same membrane as for the concentration step. However, if desired, the diafiltration step may be effected using a separate membrane with a different molecular weight cut-off, such as a membrane having a molecular weight cut-off in the range of about 3,000 to about 100,000 daltons, preferably about 5,000 to about 10,000 daltons, having regard to different membrane materials and configuration.

An antioxidant may be present in the diafiltration medium during at least part of the diafiltration step. The antioxidant may be any convenient antioxidant, such as sodium sulfite or ascorbic acid. The quantity of antioxidant employed in the diafiltration medium depends on the materials employed and may vary from about 0.01 to about 1 wt %, preferably about 0.05 wt %. The antioxidant serves to inhibit oxidation of phenolics present in the concentrated canola protein isolate solution.

The concentration step and the diafiltration step may be effected at any convenient temperature, generally about 20° to about 60° C., preferably about 20 to about 30° C., and for the period of time to effect the desired degree of concentration. The temperature and other conditions used to some degree depend upon the membrane equipment used to effect the concentration and the desired protein concentration of the solution.

The concentrated and optionally diafiltered protein solution may be subject to a further defatting operation, if required, as described in U.S. Pat. Nos. 5,844,086 and 6,005,076.

The concentrated and optionally diafiltered protein solution may be subject to a colour removal operation as an alternative to the colour removal operation described above. Powdered activated carbon may be used herein as well as granulated activated carbon (GAC). Another material which may be used as a colour absorbing agent is polyvinyl pyrrolidone.

The colour absorbing agent treatment step may be carried out under any convenient conditions, generally at the ambient temperature of the canola protein solution. For powdered activated carbon, an amount of about 0.025% to about 5% w/v, preferably about 0.05% to about 2% w/v, may be used. Where polyvinylpyrrolidone is used as the colour absorbing agent, an amount of about 0.5% to about 5% w/v, preferably about 2% to about 3% w/v, may be used. The colour absorbing agent may be removed from the canola protein solution by any convenient means, such as by filtration.

The concentrated and optionally diafiltered canola protein solution resulting from the optional colour removal step may be subjected to pasteurization to reduce the microbial load. Such pasteurization may be effected under any desired pasteurization conditions. Generally, the concentrated and optionally diafiltered canola protein solution is heated to a temperature of about 55° to about 70° C., preferably about 60° to about 65° C., for about 10 to about 15 minutes, preferably about 10 minutes. The pasteurized concentrated canola protein solution then may be cooled for further processing as described below, preferably to a temperature of about 25° to about 40° C.

Depending on the temperature employed in the concentration step and optional diafiltration step and whether or not a pasteurization step is effected, the concentrated protein solution may be warmed to a temperature of at least about 20°, and up to about 60° C., preferably about 25° to about 40° C., to decrease the viscosity of the concentrated protein solution to facilitate performance of the subsequent dilution step and micelle formation. The concentrated protein solution should not be heated beyond a temperature above which micelle formation does not occur on dilution by chilled water.

The concentrated protein solution resulting from the concentration step and optional diafiltration step, optional colour removal step, optional defatting step and optional pasteurization step then is diluted to effect micelle formation by adding the concentrated protein solution into a body of water having the volume required to achieve the degree of dilution desired. Depending on the proportion of canola protein desired to be obtained by the micelle route and the proportion from the supernatant, the degree of dilution of the concentrated protein solution may be varied. With lower dilution levels, in general, a greater proportion of the canola protein remains in the aqueous phase.

When it is desired to provide the greatest proportion of the protein by the micelle route, the concentrated protein solution is diluted by about 5 fold to about 25 fold, preferably by about 10 fold to about 20 fold.

The body of water into which the concentrated protein solution is fed has a temperature of less than about 15° C., generally about 3° C. to about 15° C., preferably less than about 11° C., since improved yields of protein isolate in the form of protein micellar mass are attained with these colder temperatures at the dilution factors used.

The dilution of the concentrated protein solution and consequential decrease in ionic strength causes the formation of a cloud-like mass of highly associated protein molecules in the form of discrete protein droplets in micellar form. The protein micelles are allowed to settle to form an aggregated, coalesced, dense, amorphous, sticky, gluten-like protein micellar mass. The settling may be assisted, such as by centrifugation. Such induced settling decreases the liquid content of the protein micellar mass, thereby decreasing the moisture content generally from about 70% by weight to about 95% by weight to a value of generally about 50% by weight to about 80% by weight of the total micellar mass. Decreasing the moisture content of the micellar mass in this way also decreases the occluded salt content of the micellar mass, and hence the salt content of dried isolate.

In a batch operation, the batch of concentrated protein solution is added to a static body of chilled water having the desired volume, as discussed above. The dilution of the concentrated protein solution and consequential decrease in ionic strength causes the formation of a cloud-like mass of highly associated protein molecules in the form of discrete protein droplets in micellar form. In the batch procedure, the protein micelles are allowed to settle in the body of chilled water to form an aggregated, coalesced, dense, amorphous sticky gluten-like protein micellar mass (PMM). The settling may be assisted, such as by centrifugation. Such induced settling decreases the liquid content of the protein micellar mass, thereby decreasing the moisture content generally from about 70% by weight to about 95% by weight to a value of generally about 50% by weight to about 80% by weight of the total micellar mass. Decreasing the moisture content of the micellar mass in this way also decreases the occluded salt content of the micellar mass, and hence the salt content of dried isolate.

Alternatively, the dilution operation may be carried out continuously by continuously passing the concentrated protein solution to one inlet of a T-shaped pipe, while the diluting water is fed to the other inlet of the T-shaped pipe, permitting mixing in the pipe. The diluting water is fed into the T-shaped pipe at a rate sufficient to achieve the desired degree of dilution of the concentrated protein solution.

The mixing of the concentrated protein solution and the diluting water in the pipe initiates the formation of protein micelles and the mixture is continuously fed from the outlet from the T-shaped pipe into a settling vessel, from which, when full, supernatant is permitted to overflow. The mixture preferably is fed into the body of liquid in the settling vessel in a manner which minimizes turbulence within the body of liquid.

In the continuous procedure, the protein micelles are allowed to settle in the settling vessel to form an aggregated, coalesced, dense, amorphous, sticky, gluten-like protein micellar mass (PMM) and the procedure is continued until a desired quantity of the PMM has accumulated in the bottom of the settling vessel, whereupon the accumulated PMM is removed from the settling vessel. In lieu of settling by sedimentation, the PMM may be separated continuously by centrifugation.

The combination of process parameters of concentrating of the protein solution to a preferred protein content of at least about 200 g/L and the use of a dilution factor of about 10 to about 20, result in higher yields, often significantly higher yields, in terms of recovery of protein in the form of protein micellar mass from the original meal extract, and much purer isolates in terms of protein content than achieved using any of the known prior art protein isolate forming procedures discussed in the aforementioned US patents.

By the utilization of a continuous process for the recovery of canola protein isolate as compared to the batch process, the initial protein extraction step can be significantly reduced in time for the same level of protein extraction and significantly higher temperatures can be employed in the extraction step. In addition, in a continuous operation, there is less chance of contamination than in a batch procedure, leading to higher product quality and the process can be carried out in more compact equipment.

The settled isolate, in the form of an amorphous, aggregated, sticky, gelatinous, gluten-like protein mass, termed “protein micellar mass”, or PMM, is separated from the residual aqueous phase or supernatant, such as by decantation of the residual aqueous phase from the settled mass or by centrifugation. The PMM may be used in the wet form or may be dried, by any convenient technique, such as spray drying, freeze drying or vacuum drum drying, to a dry form. The dry PMM has a high protein content, at least about 90 wt % protein, preferably at least about 100 wt %, (calculated as N×6.25) d.b., and is substantially undenatured (as determined by differential scanning calorimetry). The dry PMM has a low residual fat content which may be below about 1 wt %.

The supernatant from the PMM formation and settling step contains significant amounts of canola protein, not precipitated in the dilution step.

The supernatant from the dilution step, following removal of the PMM, may be concentrated to increase the protein concentration thereof. Such concentration is effected using any convenient selective membrane technique, such as ultrafiltration, using membranes with a suitable molecular weight cut-off permitting low molecular weight species, including salt, carbohydrates, pigments and other low molecular weight materials extracted from the source material, to pass through the membrane, while retaining a significant proportion of the canola protein in the solution. Ultrafiltration membranes having a molecular weight cut-off of about 3,000 to about 100,000 Daltons, preferably about 5,000 to about 10,000 Daltons, having regard to differing membrane materials and configurations, may be used. Concentration of the supernatant in this way also reduces the volume of liquid required to be dried to recover the protein, and hence the energy required for drying. The supernatant generally is concentrated to a protein content of about 100 to 400 g/L, preferably about 200 to about 300 g/L, prior to drying.

The concentrated supernatant may be dried by any convenient technique, such as spray drying, freeze drying or vacuum drum drying, to a dry form to provide a further canola protein isolate. Such further canola protein isolate has a high protein content, usually in excess of about 90 wt % protein (calculated as Kjeldahl N×6.25) and is substantially undenatured (as determined by differential scanning calorimetry). If desired, the wet PMM may be combined with the concentrated supernatant prior to drying the combined protein streams by any convenient technique to provide a combined canola protein isolate. The combined canola protein isolate has a high protein content, in excess of about 90 wt % (calculated as Kjeldahl N×6.25) and is substantially undenatured (as determined by differential scanning calorimetry).

Alternatively, the supernatant from the separation of the PMM may be processed by alternative procedures to recover further canola protein isolate therefrom. For example, as described in copending U.S. patent application Ser. No. 12/213,500 filed Jun. 20, 2008, assigned to the assignee hereof and the disclosures of which are incorporated herein by reference, the supernatant, which first may be partially concentrated or concentrated, may be heat treated to precipitate 7S protein therefrom prior to recovery of the canola protein isolate from the heat-treated solution. As also described in copending U.S. patent application Ser. No. 12/213,500, the supernatant may be subjected to isoelectric precipitation to deposit 7S protein, prior to recovery of the canola protein isolate from the resulting solution.

In another alternative as described in US Provisional Patent Application No. 61/136,193 filed Aug. 18, 2008, assigned to the assigned herein and the disclosures of which are incorporated herein by reference (U.S. patent application Ser. No. ______ filed ______, WO ______), the supernatant, which may first be partially concentrated or concentrated, is subjected to treatment by a calcium salt, preferably calcium chloride, prior to recovery of the canola protein isolate.

Additionally, as described in U.S. Provisional Patent Application No. 61/136,208 filed Aug. 19, 2008, assigned to the assignee hereof and the disclosures of which are incorporated herein by reference (U.S. patent application Ser. No. ______ filed ______, Wo ______), the PMM may be processed to provide a soluble canola protein isolate.

In another alternative procedure, a portion only of the concentrated supernatant may be mixed with at least part of the PMM and the resulting mixture dried. The remainder of the concentrated supernatant may be dried as any of the remainder of the PMM. Further, dried PMM and dried supernatant also may be dry mixed in any desired relative proportions.

By operating in this manner, a number of canola protein isolates may be recovered, in the form of dried PMM, dried supernatant and dried mixtures of various proportions by weight of PMM and supernatant, generally from about 5:95 to about 95:5 by weight, which may be desirable for attaining differing functional and nutritional properties.

### EXAMPLES

### Example 1

This Example describes the production of a novel canola protein isolate in accordance with one embodiment of the invention.

‘a’ kg of canola seed was passed through a grinder to fully grind the seed. ‘b’ kg of ground seed was added to ‘c’ L of ‘d’ M NaCl solution at ambient temperature and agitated for 30 minutes to provide an aqueous protein solution. The residual canola seed material was removed and the resulting protein solution was partially clarified by centrifugation to produce ‘e’ L of partially clarified protein solution having a protein content of ‘f’ % by weight. The partially clarified protein solution was defatted with a cream separator and then filtered to further clarify resulting in a solution of volume ‘g’ L having a protein content of ‘h’ % by weight.

A ‘i’ L aliquot of the protein extract solution was reduced in volume to ‘j’ L by concentration on a polyethersulfone (PES) membrane having a molecular weight cutoff of ‘k’ Daltons and then diafiltered with ‘l’ volumes of ‘m’ M NaCl solution on the same membrane. The diafiltered retentate was then pasteurized at 60° C. for 1 minute. The resulting ‘n’ kg of pasteurized concentrated protein solution had a protein content of ‘o’ % by weight.

The concentrated solution at ‘p’ ° C. was diluted ‘q’ into cold reverse osmosis (RO) purified water having a temperature ‘r’ ° C. A white cloud formed immediately and was allowed to settle. The upper diluting water was removed and the precipitated, viscous, sticky mass (PMM) was recovered by centrifugation in a yield of ‘s’ wt % of the filtered protein solution. The dried PMM derived protein was found to have a protein content of ‘t’ % (N×6.25) d.b. The product was given a designation ‘u’ C300.

The parameters ‘a’ to ‘u’ for two runs are set forth in the following Table I:

TABLE I

| u | BW-CC089-B19-08A | BW-EC091-B21-08A |
|---|---|---|
| a | 22.5 | 22.5 |
| b | 21.4 | 21.82 |
| c | 150 | 150 |
| d | 0.15 | 0.15 |
| e | 145.7 | 136.6 |
| f | 1.30 | 1.12 |
| g | 148 | 108 |
| h | 0.92 | 0.76 |
| i | 148 | 108 |
| j | 5 | 5 |
| k | 100,000 | 100,000 |
| l | 5 | 5 |
| m | 0.15 | 0.15 |
| n | 6 | 5.16 |
| o | 18.46 | 14.81 |
| p | 30 | 30 |
| q | 1:10 | 1:10 |
| r | 2.7 | 3 |
| s | 52.2 | 42.7 |
| t | 101.52 | 97.00 |

‘v’ L supernatant was heated to 80° C. for 10 minutes and then centrifuged to remove precipitated protein. The centrifuged heat treated supernatant was then reduced in volume from ‘w’ L to ‘x’ L by ultrafiltration using a polyethersulfone (PES) membrane having a molecular weight cut-off of ‘y’ Daltons and then the concentrate was diafiltered on the same membrane with ‘z’ volumes of pH 3 RO water. The diafiltered concentrate contained ‘aa’ % protein by weight. With the additional protein recovered from the supernatant, the overall protein recovery of the filtered protein solution was ‘ab’ wt %. The concentrate was spray dried to form a final product given designation ‘u’ C200HS and had a protein content of ‘ac’ % (N×6.25) d.b. The parameters ‘u’ to ‘ac’ for two runs are set forth in the following Table II:

TABLE II

| u | BW-CC089-B19-08A | BW-EC091-B21-08A |
|---|---|---|
| v | 60 | 55 |
| w | 46 | 50 |
| x | 5 | 5 |
| y | 10,000 | 10,000 |
| z | 5 | 5 |
| aa | 5.23 | 3.85 |
| ab | 67.6 | 64.6 |
| ac | 99.33 | 101.49 |

### Example 2

This Example describes the production of a novel canola protein isolate in accordance with one embodiment of the invention.

‘a’ kg of myrosinase inactivated canola seed was passed through a grinder to fully grind the seed. ‘b’ kg of ground seed was added to ‘c’ L of ‘d’ M NaCl solution at ambient temperature and agitated for 30 minutes to provide an aqueous protein solution. The residual canola seed material was removed and the resulting protein solution was partially clarified by centrifugation to produce a partially clarified protein solution having a protein content of ‘e’ % by weight. The partially clarified protein solution was defatted with a cream separator and then filtered to further clarify resulting in a solution of volume ‘f’ L having a protein content of ‘g’ % by weight.

A ‘h’ L aliquot of the protein extract solution was reduced to ‘i’ kg by concentration on a polyvinylidene fluoride (PVDF) membrane having a molecular weight cutoff of ‘j’ daltons. The retentate was then pasteurized at approximately 62° C. for 10 minutes. The resulting ‘k’ kg of pasteurized concentrated protein solution had a protein content of ‘l’ % by weight.

The concentrated solution at ‘m’ ° C. was diluted ‘n’ into cold RO water having a temperature ‘o’ ° C. A white cloud formed immediately and was allowed to settle. The upper diluting water was removed and the precipitated, viscous, sticky mass (PMM) was recovered either by centrifugation in a yield of ‘p’ wt % of the filtered protein solution. The dried PMM derived protein was found to have a protein content of ‘q’ % (N×6.25) d.b. The product was given a designation ‘r’ C300.

The parameters ‘a’ to ‘r’ are set forth in the following Table III:

TABLE III

| r | BW-EH066-H09-06A |
|---|---|
| a | 22.5 |
| b | 18 |
| c | 120 |
| d | 0.15 |
| e | 0.88 |
| f | 106 |
| g | 0.80 |
| h | 106 |
| i | 3.69 |
| j | 30,000 |
| k | 3.52 |
| I | 14.03 |
| m | 30 |
| n | 1:15 |
| o | 2.0 |
| p | 31.8 |
| q | 99.29 |

‘s’ L of supernatant was heated to approximately 87° C. for 5 minutes and then centrifuged to remove precipitated protein. The centrifuged heat treated supernatant was then reduced from ‘t’ L to ‘u’ kg by ultrafiltration using a polyethersulfone (PES) membrane having a molecular weight cut-off of ‘v’ Daltons. The retentate contained ‘w’ % protein by weight. With the additional protein recovered from the supernatant, the overall protein recovery of the filtered protein solution was ‘x’ wt %. The retentate was spray dried to form a final product with a protein content of ‘y’ % (N×6.25) d.b. and given designation ‘r’ C200HS.

The parameters ‘r’ to ‘y’ are set forth in the following Table IV:

TABLE IV

| r | BW-EH066-H09-06A |
|---|---|
| s | 53.2 |
| t | 49 |
| u | 4 |
| v | 10,000 |
| w | 3.37 |
| x | 47.1 |
| y | 90.34 |

### Example 3

This Example describes the production of a canola protein isolate using meal prepared from the myrosinase inactivated canola seed used in Example 2.

‘a’ kg of myrosinase inactivated canola meal was added to ‘b’ L of ‘c’ M NaCl solution at ambient temperature and agitated for 30 minutes to provide an aqueous protein solution. The residual canola meal was removed and the resulting protein solution was partially clarified by centrifugation to produce ‘d’ L of partially clarified protein solution having a protein content of ‘e’ % by weight. This solution was then filtered to further clarify resulting in a solution of volume ‘f’ L having a protein content of ‘g’ % by weight.

A ‘h’ L aliquot of the protein extract solution was reduced to ‘i’ kg by concentration on a PVDF (polyvinylidene fluoride) membrane having a molecular weight cutoff of ‘j’ Daltons. The retentate was then pasteurized at approximately 63° C. for 10 minutes. The resulting ‘k’ kg of pasteurized concentrated protein solution had a protein content of ‘1’ % by weight.

The concentrated solution at ‘m’ ° C. was diluted ‘n’ into cold RO water having a temperature ‘o’ ° C. A white cloud formed immediately and was allowed to settle. The upper diluting water was removed and the precipitated, viscous, sticky mass (PMM) was recovered by centrifugation in a yield of ‘p’ wt % of the filtered protein solution. The dried PMM derived protein was found to have a protein content of ‘q’ % (N×6.25) d.b. The product was given a designation ‘r’ C300.

The parameters ‘a’ to ‘r’ for three runs are set forth in the following Table V:

TABLE V

| r | BW-SD062-G19-06A |
|---|---|
| a | 15 |
| b | 150 |
| c | 0.15 |
| d | 115.1 |
| e | 1.99 |
| f | 110 |
| g | 1.47 |
| h | 110 |
| i | 4.89 |
| j | 30,000 |
| k | 4.8 |
| l | 23.9 |
| m | 26 |
| n | 1:20 |
| o | 2 |
| p | 40.7 |
| q | 101.87 |

‘s’ L of supernatant was heated to approximately 85° C. for 8 minutes and then centrifuged to remove precipitated protein. The ‘t’ L of centrifuged heat treated supernatant was then reduced to ‘u’ kg by ultrafiltration using a polyethersulfone (PES) membrane having a molecular weight cut-off of ‘v’ Daltons. The retentate contained ‘w’ % protein by weight. With the additional protein recovered from the supernatant, the overall protein recovery of the filtered protein solution was ‘x’ wt %. The retentate was spray dried to form a final product with a protein content of ‘y’ % (N×6.25) d.b. and given designation ‘r’ C200HS.

The parameters ‘r’ to ‘y’ for two runs are set forth in the following Table VI:

TABLE VI

| r | BW-SD062-G19-08A |
|---|---|
| s | 112 |
| t | 92 |
| u | 4.1 |
| v | 10,000 |
| w | 5.91 |
| x | 55.6 |
| y | 96.68 |

The colors of the dry products produced in the above examples were analyzed using a HunterLab ColorQuest XE instrument operated in reflectance mode. The results are shown in Table VII.

TABLE VII
Color results for dried product from Examples 2 and 3:

| | Hunter Lab | | |
|---|---|---|---|
| | Color Readings | | |
| | L* | a* | b* |
| BW-EH066-H09-06A C200HS | 86.54 | −2.03 | 15.57 |
| BW-SD062-G19-06A C200HS | 82.65 | −1.52 | 15.97 |
| BW-EH066-H09-06A C300 | 78.71 | −2.23 | 27.51 |
| BW-SD062-G19-06A C300 | 76.31 | −2.22 | 25.89 |

Canola protein isolates prepared from ground seed were found to be lighter (higher L* value) than the equivalent products produced from canola meal prepared from the same seed. The C200HS prepared from seed was a little greener than the product prepared from meal, whereas the C300 products had very similar a* values, regardless of the starting material. The C200HS prepared from ground seed was less yellow than the C200HS prepared from meal, but the trend was reversed for the C300 products. Samples with higher L* values are generally considered more acceptable as the “L*” value is an indication of whiteness. A maximum value of 100 indicates a white sample while a minimum value of 0 would indicate a black sample.

### SUMMARY OF THE DISCLOSURE

In summary of this disclosure, the present invention is concerned with the production of a canola protein isolate from canola oil seeds in which there is no initial removal of oil from the seeds. Modifications are possible within the scope of the invention.

What we claim is:

1. A process for the preparation of a canola protein isolate, which comprises:
grinding canola oil seeds,
extracting the ground canola oil seeds with an aqueous extracting medium to solubilize canola protein and fats in the ground canola oil seeds to form an aqueous canola protein solution,
separating the aqueous canola protein solution from residual ground canola oil seeds,
defatting the aqueous canola protein solution,
clarifying the defatted aqueous canola protein solution,
concentrating the clarified aqueous canola protein solution while maintaining the ionic strength substantially constant to form a concentrated canola protein solution,
diluting the concentrated protein solution into chilled water to cause the formation of canola protein micelles,
collecting the canola protein micelles as a protein micellar mass, and
drying the protein micellar mass to form a canola protein isolate having a protein content of at least about 90 wt % (N×6.25) d.b.

2. The process of claim 1, wherein said aqueous extracting medium is an aqueous salt solution having an ionic strength of at least about 0.05 M with a pH of about 5 to about 6.8 to form a canola protein solution having a concentration of about 3 to about 40 g/L.

3. The process of claim 2 wherein an antioxidant is present in the aqueous extracting medium.

4. The process of claim 1 wherein said defatting step is effected by chilling the canola protein solution to a temperature of about 3° to about 7° C. and removing fat that separates from the canola protein solution.

5. The process of claim 4 wherein, following the defatting step, the separated aqueous canola protein solution is subjected to a colour removal step.

6. The process of claim 1 wherein said aqueous canola protein solution is concentrated to a protein concentration of about 50 to about 250 g/L.

7. The process of claim 6 wherein the concentrated canola protein solution is subjected to a diafiltration step to provide a concentrated and diafiltered canola protein solution.

8. The process of claim 7 wherein an antioxidant is present during at least part of the diafiltration operation.

9. The process of claim 7 wherein the concentrated and diafiltered canola protein solution is subjected to a colour removal operation.

10. The process of claim 7 wherein the concentrated and diafiltered canola protein solution is subjected to a pasteurization step.

11. The process of claim 1 wherein said dilution step is effected by diluting the concentrated protein solution by about 5 fold to about 25 fold at a temperature of less than about 15° C.

12. The process of claim 1 wherein the supernatant from the collection of the protein micellar mass is processed to form further canola protein isolate having a protein content of at least about 90 wt % (N×6.25) d.b.
